(19) United States
(12) Patent Application Publication
(10) Pub. No.: US 2006/0110449 A1
(43) Pub. Date: May 25, 2006

(54) Pharmaceutical composition

(76) Inventors: Richard R. Lorber, Scotch Plains, NJ (US); Heribert W. Staudinger, Union, NJ (US); Robert E. Ward, Summit, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/257,348

(22) Filed: Oct. 24, 2005

Related U.S. Application Data
(60) Provisional application No. 60/622,507, filed on Oct. 27, 2004.
(60) Provisional application No. 60/621,783, filed on Oct. 25, 2004.

(51) Int. Cl. A61K 31/473; A61K 31/24; A61K 9/20

(52) U.S. Cl. 424/464; 514/290; 514/540

(57) ABSTRACT

The present invention relates to formulations useful for treating respiratory disorders associated with the production of mucus glycoprotein, skin disorders, and allergic conjunctivitis while substantially reducing adverse effects associated with the administration of non-selective anti-cholinergic agents and methods of use thereof.

### CROSS REFERENCE TO RELATED APPLICATION

This application claims benefit of priority to U.S. Patent Application 60/622,507, filed Oct. 27, 2004, the entirety of which is incorporated by reference as if set forth fully herein.

### FIELD OF THE INVENTION

The present invention relates to formulations useful for treating respiratory disorders associated with the production of mucus glycoprotein, skin disorders, and allergic conjunctivitis while substantially reducing adverse effects associated with the administration of non-selective anti-cholinergic agents. In particular, the formulations include a selective muscarinic acetylcholine receptor subtype M1 and/or M3 antagonist in combination with one or more pharmaceutically active agents.

### BACKGROUND OF THE INVENTION

Cholinergic receptors are divided into two major classes: nicotinic acetylcholine receptors and muscarinic acetylcholine receptors based on their responsiveness to nicotine and muscarine, respectively. Unlike nicotinic acetylcholine receptors which are ion channels, muscarinic acetylcholine receptors belong to the superfamily of G-protein coupled receptors that activate ionic channels through a second messenger cascade. Muscarinic acetylcholine receptors are further divided into subtypes M1-M5 characterized by their cellular actions, pharmacology, and molecular biology.

Likewise, anti-cholinergic agents are classified as either anti-nicotinic acetylcholine agents or anti-muscarinic acetylcholine agents based on whether nicotinic acetylcholine receptors or muscarinic acetylcholine receptors, respectively, are targeted. Moreover, anti-muscarinic acetylcholine agents are further classified based on the subtype of muscarinic acetylcholine receptor (M1-M5) that is targeted.

Of the several subtypes of muscarinic acetylcholine receptor, subtypes M1 and M3 play important roles in the regulation of glandular secretion and vasomotor tone in human nasal mucosa [Okayama et al., Am J Respir Cell Mol Biol, 8(2):176-187 (1993); Mullol etal., J Appl Physiol, 73(5):2069-2073 (1992)]. In particular, it has been suggested that muscarinic acetylcholine receptor subtype M3 has the predominant effect on mucus glycoprotein secretion from human nasal mucosa [Mullol et al., J Appl Physiol, 73(5):2069-2073 (1992)]. In contrast, muscarinic acetylcholine receptor subtype M2 appears to have no effect on mucus glycoprotein secretion [Mullol et al., J Appl Physiol, 73(5):2069-2073 (1992)], but exclusively mediates the negative chronotropic effects on heart rate following vagal stimulation or administration of muscarinic agonists [Wess, Annu Rev Pharmacol Toxicol, 44:423-450 (2004)].

Numerous respiratory disorders are associated with the production and secretion of mucus glycoprotein (i.e., high-molecular weight glycoconjugates released from submucosal glands and epithelial goblet cells in the respiratory tract). Previous therapies focused on using non-sedating formulations to treat such respiratory disorders. For example, Weinstein and Weinstein (U.S. Pat. No. 6,086,914) describe methods of treating allergic rhinitis using an anti-cholinergic agent with a limited capacity to pass across lipid membranes, such as the blood-brain barrier, in combination with an antihistamine that is limited in both sedating and anti-cholinergic properties. Such non-sedating formulations however, employ non-selective anti-cholinergic agents (e.g., methscopolamine nitrate, glycopyrrolate, and atropine sulfate) that result in adverse effects including stimulation of the cardiovascular system (e.g., tachycardia).

Unlike previous therapies which employ non-selective anti-cholinergic agents that result in adverse effects including stimulation of the cardiovascular system (e.g., tachycardia), there is a need for formulations useful for treating respiratory disorders associated with the production and secretion of mucus glycoprotein while reducing adverse effects (e.g., stimulation of the cardiovascular system) of previous therapeutics.

### SUMMARY OF THE INVENTION

The present invention provides formulations useful for treating respiratory disorders associated with the production of mucus glycoprotein, skin disorders, and allergic conjunctivitis while reducing adverse effects (e.g., stimulation and/or depression of the central nervous system and/or stimulation of the cardiovascular system). In particular, the present inventors believe that formulations that include a selective muscarinic acetylcholine receptor subtype M1 and/or M3 antagonist in combination with one or more pharmaceutically active agents will reduce adverse effects (e.g., stimulation and/or depression of the central nervous system and/or stimulation of the cardiovascular system) associated with the use of non-selective acetylcholine receptor agents. In addition, the present inventors envisage that the present formulations will permit a lower dose of one or more pharmaceutically active agents to be administered to achieve a therapeutic effect than would otherwise be required, thereby reducing adverse effects associated with the dosage administered. Furthermore, in one embodiment, wherein the formulation comprises the selective muscarinic acetylcholine receptor subtype M1 and M3 antagonist oxybutynin in combination with the antihistamine desloratadine, the present inventors believe that the combination will be stable despite desloratadine's known chemical reactivity and will provide formulations with an improved degradation profile.

The present invention also provides methods using these formulations for treating respiratory disorders associated with the production of mucus glycoprotein, skin disorders, and allergic conjunctivitis while reducing adverse effects (e.g., stimulation and/or depression of the central nervous system and/or stimulation of the cardiovascular system) of previous therapeutics.

The present invention provides formulations comprising a selective muscarinic acetylcholine receptor subtype M1 and/or M3 antagonist in combination with one or more pharmaceutically active agents.

In a preferred embodiment, the selective muscarinic acetylcholine receptor subtype M1 and/or M3 antagonist is glycopyrronium bromide, telenzepine, pirenzepine, oxybutynin, desethyloxybutynin, himbacine, AQ-RA 741, darifenacin, hexahydrosila-difenidol, p-flurohexahydro-sila-difenidol (p-F-HHSiD)), 4-diphenylacetoxy-N-methylpiperidine (4-DAMP) methiodide, or 4-DAMP methobromide, or a pharmaceutically acceptable salt of any of these agents, or a combination of one or more of these agents.

In one embodiment, the selective muscarinic acetylcholine receptor subtype M1 and/or M3 antagonist is a selective muscarinic acetylcholine receptor subtype M1 antagonist. Preferably, the selective muscarinic acetylcholine receptor subtype M1 antagonist is glycopyrronium bromide, telenzepine, or pirenzepine, or a pharmaceutically acceptable salt of any of these agents, or a combination of one or more of these agents. More preferably, the selective muscarinic acetylcholine receptor subtype M1 antagonist is pirenzepine or a pharmaceutically acceptable salt thereof. Preferably, the unit dosage form (single or divided dosage form as is known to one of skill in the art) of pirenzepine is in the range of about 2.5 mg to about 250 mg. More preferably, the unit dosage form of pirenzepine is in the range of about 100 mg to about 150 mg. Still more preferably, the unit dosage form of pirenzepine is about 50 mg administered b.i.d or t.i.d.

In another embodiment, the selective muscarinic acetylcholine receptor subtype M1 and/or M3 antagonist is a selective muscarinic acetylcholine receptor subtype M3 antagonist. Preferably, the selective muscarinic acetylcholine receptor subtype M3 antagonist is himbacine, AQ-RA 741, darifenacin, hexahydrosila-difenidol, p-flurohexahydro-sila-difenidol (p-F-HHSiD)), 4-diphenylacetoxy-N-methylpiperidine (4-DAMP) methiodide, or 4-DAMP methobromide, or a pharmaceutically acceptable salt of any of these agents, or a combination of one or more of these agents. More preferably, the selective muscarinic acetylcholine receptor subtype M3 antagonist is darifenacin or a pharmaceutically acceptable salt thereof.

In another embodiment, the selective muscarinic acetylcholine receptor subtype M1 and/or M3 antagonist is a selective muscarinic acetylcholine receptor subtype M1 and M3 antagonist. Preferably, the selective muscarinic acetylcholine receptor subtype M1 and M3 antagonist is oxybutynin, desethyloxybutynin, or a pharmaceutically acceptable salt of any of these agents, or a combination of one or more of these agents. More preferably, the selective muscarinic acetylcholine receptor subtype M1 and M3 antagonist is oxybutynin, desethyloxybutynin, or a pharmaceutically acceptable salt of any of these agents. In one embodiment, one or more pharmaceutically active agents is an antihistamine. Preferably, the antihistamine is azatadine, azelastine, acrivastine, brompheniramine, cetirizine, chlorpheniramine, clemastine, cyclizine, carebastine, cyproheptadine, carbinoxamine, desloratadine, doxylamine, dimethindene, ebastine, epinastine, efletirizine, fexofenadine, hydroxyzine, ketotifen, loratadine, levocabastine, levocetirizine, mizolastine, mequitazine, mianserin, noberastine, meclizine, norastemizole, picumast, pyrilamine, promethazine, tripelennamine, temelastine, trimeprazine, triprolidine, thioperamide, impromidine, burimamide, clobenpropit, impentamine, mifetidine, S-sopromidine, R-sopromidine, 3-(imidazol-4-yl)-propylguanidine (SKF-91486), 3-[(4-chlorophenyl)methyl]-5-[2-(1 H-imidazol-4yl)ethyl] 1,2,3-oxadiazole (GR-175737), 4-(1-cyclohexylpentanoyl-4-piperidyl) 1H-imidazole (GT-2016), 2-{[2-[4(5)-imidazolyl]ethyl]thio}-5-nitropyridine (UCL-1199), or clozapine, or a pharmaceutically acceptable salt of any of these agents, or a combination of one or more of these agents. More preferably, the antihistamine is desloratadine or a pharmaceutically acceptable salt thereof.

In still yet another embodiment, one or more pharmaceutically active agents is a decongestant. Preferably, the decongestant is a histamine H₃ receptor antagonist (e.g., thioperamide, impromidine, burimamide, clobenpropit, impentamine, mifetidine, S-sopromidine, R-sopromidine, 3-(imidazol-4-yl)-propylguanidine (SKF-91486), 3-[(4-chlorophenyl)methyl]-5-[2-(1 H-imidazol-4yl)ethyl] 1,2,3-oxadiazole (GR-175737), 4-(1-cyclohexylpentanoyl-4-piperidyl) 1H-imidazole (GT-2016), 2-{[2-[4(5)-imidazolyl]ethyl]thio}-5-nitropyridine (UCL-1199), clozapine), levmetamfetamine, ephedrine, ephedrine hydrochloride, ephedrine sulfate, naphazoline hydrochloride, oxymetazoline hydrochloride, phenylephrine hydrochloride, propylhexedrine, xylometazoline hydrochloride, phenylpropanolamine, phenylephrine, or pseudoephedrine, or a pharmaceutically acceptable salt of any of these agents, or a combination of one or more of these agents. More preferably, the decongestant is phenylephrine or pseudoephedrine, or a pharmaceutically acceptable salt of any of these agents.

In yet other embodiments, one or more pharmaceutically active agents is a corticosteroid; an expectorant; a composition to relieve oropharyngeal discomfort; a P2Y₂ receptor antagonist; a non-steroidal anti-inflammatory agent; a leukotriene antagonist; a syk kinase inhibitor; or a 5-lipoxygenase inhibitor.

In one preferred embodiment, the formulation comprises a therapeutically effective amount of darifenacin or a pharmaceutically acceptable salt thereof in combination with a therapeutically effective amount of desloratadine or a pharmaceutically acceptable salt thereof.

In another preferred embodiment, the formulation comprises a therapeutically effective amount of oxybutynin or a pharmaceutically acceptable salt thereof in combination with a therapeutically effective amount of desloratadine or a pharmaceutically acceptable salt thereof.

In one embodiment, the pharmaceutically acceptable salt of oxybutynin is prepared from a pharmaceutically acceptable acid addition salt selected from the group consisting of acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, and p-toluene sulfonic acid. In a more preferred embodiment, the pharmaceutically acceptable salt of oxybutynin is oxybutynin chloride.

In one embodiment, the therapeutically effective amount of oxybutynin or a pharmaceutically acceptable salt thereof is a unit dosage form (single or divided dosage form as is known to one of skill in the art) in a range from about 0.1 mg to about 1 g administered q.d. Preferably, for administration orally, the therapeutically effective amount of oxybutynin or a pharmaceutically acceptable salt thereof is a unit dosage form in a range from about 1 mg to about 1 g administered q.d., more preferably, in a range from about 25 mg to about 700 mg administered q.d. Preferably, for administration by oral or intranasal inhalation, the therapeutically effective amount of oxybutynin or a pharmaceutically acceptable salt thereof is a unit dosage form in a range from about 0.1 mg to about 100 mg administered q.d. Yet more preferably, the therapeutically effective amount of oxybutynin or a pharmaceutically acceptable salt thereof is a unit dosage form in a range from about 1.25 mg to about 30 mg administered q.d (e.g., about 1.25 mg, about 2.5 mg, about 5 mg, about 10 mg, about 15 mg, about 20 mg, about 25 mg, about 30 mg administered q.d.). Still more preferably, the therapeutically effective amount of oxybutynin or a pharmaceutically acceptable salt thereof is a unit dosage form which is about 1.25 mg, about 2.5 mg, about 3 mg, about 3.5 mg, about 4 mg, about 4.5 mg, or about 5 mg administered b.i.d., t.i.d., or q.i.d. Yet still more preferably, the therapeutically effective amount of oxybutynin or a pharmaceutically acceptable salt thereof is a unit dosage form which is about 2.5 mg or about 5 mg administered b.i.d.

In one embodiment, the therapeutically effective amount of desloratadine or a pharmaceutically acceptable salt thereof is a unit dosage form (single or divided dosage form as is known to one of skill in the art) in a range from about 1.25 mg to about 45 mg administered q.d. (e.g., about 2 mg, about 4 mg, about 6 mg, about 8 mg, about 10 mg, about 12 mg, about 14 mg, about 16 mg, about 18 mg, about 20 mg, about 22 mg, about 24 mg, about 26 mg, about 28 mg, about 30 mg, about 32 mg, about 34 mg, about 36 mg, about 38 mg, about 40 mg, about 42 mg, about 44 mg administered q.d.). Preferably, the therapeutically effective amount of desloratadine or a pharmaceutically acceptable salt thereof is a unit dosage form in a range from about 1.25 mg to about 20 mg administered q.d. More preferably, the therapeutically effective amount of desloratadine or a pharmaceutically acceptable salt thereof is a unit dosage form in a range from about 5 mg to about 10 mg administered q.d. In one embodiment, the therapeutically effective amount of desloratadine or a pharmaceutically acceptable salt thereof is a unit dosage form which is about 1.25 mg, about 2.5 mg, about 5.0 mg, about 7.5 mg, about 10.0 mg, about 12.5 mg, about 15 mg, 17.5 mg, or about 20.0 mg administered q.d.

In one embodiment, the formulation comprising a therapeutically effective amount of darifenacin or oxybutynin, or a pharmaceutically acceptable salt thereof in combination with a therapeutically effective amount of desloratadine or a pharmaceutically acceptable salt thereof further comprises one or more additional pharmaceutically active agents. Preferably, one or more additional pharmaceutically active agents is another antihistamine; a decongestant; a corticosteroid; an expectorant; a composition to relieve oropharyngeal discomfort; a P2Y₂ receptor antagonist; a non-steroidal anti-inflammatory agent; a leukotriene antagonist; a syk kinase inhibitor; or a 5-lipoxygenase inhibitor.

The present invention also provides, methods for treating a respiratory disorder associated with the production of mucus glycoprotein in a patient suffering therefrom comprising administering a therapeutically effective amount of a selective muscarinic acetylcholine receptor subtype M1 and/or M3 antagonist in combination with one or more pharmaceutically active agents.

In a preferred embodiment, the selective muscarinic acetylcholine receptor subtype M1 and/or M3 antagonist is glycopyrronium bromide, telenzepine, pirenzepine, oxybutynin, desethyloxybutynin, himbacine, AQ-RA 741, darifenacin, hexahydrosila-difenidol, p-flurohexahydro-sila-difenidol (p-F-HHSiD)), 4-diphenylacetoxy-N-methylpiperidine (4-DAMP) methiodide, or 4-DAMP methobromide, or a pharmaceutically acceptable salt of any of these agents, or a combination of one or more of these agents.

In one embodiment, the selective muscarinic acetylcholine receptor subtype M1 and/or M3 antagonist is a selective muscarinic acetylcholine receptor subtype M1 antagonist. Preferably, the selective muscarinic acetylcholine receptor subtype M1 antagonist is glycopyrronium bromide, telenzepine, or pirenzepine, or a pharmaceutically acceptable salt of any of these agents, or a combination of one or more of these agents. More preferably, the selective muscarinic acetylcholine receptor subtype Ml antagonist is pirenzepine or a pharmaceutically acceptable salt thereof. Preferably, the unit dosage form (single or divided dosage form as is known to one of skill in the art) of pirenzepine is in the range of about 2.5 mg to about 250 mg. More preferably, the unit dosage form of pirenzepine is in the range of about 100 mg to about 150 mg. Still more preferably, the unit dosage form of pirenzepine is about 50 mg administered b.i.d or t.i.d.

In another embodiment, the selective muscarinic acetylcholine receptor subtype M1 and/or M3 antagonist is a selective muscarinic acetylcholine receptor subtype M3 antagonist. Preferably, the selective muscarinic acetylcholine receptor subtype M3 antagonist is himbacine, AQ-RA 741, darifenacin, hexahydrosila-difenidol, p-flurohexahydro-sila-difenidol (p-F-HHSiD)), 4-diphenylacetoxy-N-methylpiperidine (4-DAMP) methiodide, or 4-DAMP methobromide, or a pharmaceutically acceptable salt of any of these agents, or a combination of one or more of these agents. More preferably, the selective muscarinic acetylcholine receptor subtype M3 antagonist is darifenacin or a pharmaceutically acceptable salt thereof.

In another embodiment, the selective muscarinic acetylcholine receptor subtype M1 and/or M3 antagonist is a selective muscarinic acetylcholine receptor subtype M1 and M3 antagonist. Preferably, the selective muscarinic acetylcholine receptor subtype M1 and M3 antagonist is oxybutynin, desethyloxybutynin, or a pharmaceutically acceptable salt of any of these agents, or a combination of one or more of these agents. More preferably, the selective muscarinic acetylcholine receptor subtype M1 and M3 antagonist is oxybutynin, desethyloxybutynin, or a pharmaceutically acceptable salt of any of these agents.

In yet another embodiment, one or more pharmaceutically active agents is an antihistamine. Preferably, the antihistamine is azatadine, azelastine, acrivastine, brompheniramine, cetirizine, chlorpheniramine, clemastine, cyclizine, carebastine, cyproheptadine, carbinoxamine, desloratadine, doxylamine, dimethindene, ebastine, epinastine, efletirizine, fexofenadine, hydroxyzine, ketotifen, loratadine, levocabastine, levocetirizine, mizolastine, mequitazine, mianserin, noberastine, meclizine, norastemizole, picumast, pyrilamine, promethazine, tripelennamine, temelastine, trimeprazine, triprolidine, thioperamide, impromidine, burimamide, clobenpropit, impentamine, mifetidine, S-sopromidine, R-sopromidine, 3-(imidazol-4-yl)-propylguanidine (SKF-91486), 3-[(4-chlorophenyl)methyl]-5-[2-(1H-imidazol-4yl)ethyl] 1,2,3-oxadiazole (GR-175737), 4-(1-cyclohexylpentanoyl-4-piperidyl) 1H-imidazole (GT-2016), 2-{[2-[4(5)-imidazolyl]ethyl]thio}-5-nitropyridine (UCL-1199), or clozapine, or a pharmaceutically acceptable salt of any of these agents, or a combination of one or more of these agents. More preferably, the antihistamine is desloratadine or a pharmaceutically acceptable salt thereof.

In still yet another embodiment, one or more pharmaceutically active agents is a decongestant. Preferably, the decongestant is a histamine H₃ receptor antagonist (e.g., thioperamide, impromidine, burimamide, clobenpropit, impentamine, mifetidine, S-sopromidine, R-sopromidine, 3-(imidazol-4-yl)-propylguanidine (SKF-91486), 3-[(4-chlorophenyl)methyl]-5-[2-(1H-imidazol-4yl)ethyl] 1,2,3-oxadiazole (GR-175737), 4-(1-cyclohexylpentanoyl-4-piperidyl) 1H-imidazole (GT-2016), 2-{[2-[4(5)-imidazolyl]ethyl]thio}-5-nitropyridine (UCL-1199), clozapine), levmetamfetamine, ephedrine, ephedrine hydrochloride, ephedrine sulfate, naphazoline hydrochloride, oxymetazoline hydrochloride, phenylephrine hydrochloride, propylhexedrine, xylometazoline hydrochloride, phenylpropanolamine, phenylephrine, or pseudoephedrine, or a pharmaceutically acceptable salt of any of these agents, or a combination of one or more of these agents. More preferably, the decongestant is phenylephrine or pseudoephedrine, or a pharmaceutically acceptable salt of any of these agents.

In yet other embodiments, one or more pharmaceutically active agents is a corticosteroid; an expectorant; a composition to relieve oropharyngeal discomfort; a P2Y₂ receptor antagonist; a non-steroidal anti-inflammatory agent; a leukotriene antagonist; a syk kinase inhibitor; or a 5-lipoxygenase inhibitor.

In one preferred embodiment, the method for treating a respiratory disorder associated with the production of mucus glycoprotein in a patient suffering therefrom comprises administering a therapeutically effective amount of darifenacin or a pharmaceutically acceptable salt thereof in combination with a therapeutically effective amount of desloratadine or a pharmaceutically acceptable salt thereof.

In another preferred embodiment, the method for treating a respiratory disorder associated with the production of mucus glycoprotein in a patient suffering therefrom comprises administering a therapeutically effective amount of oxybutynin or a pharmaceutically acceptable salt thereof in combination with a therapeutically effective amount of desloratadine or a pharmaceutically acceptable salt thereof.

In one embodiment, the pharmaceutically acceptable salt of oxybutynin is prepared from a pharmaceutically acceptable acid addition salt selected from the group consisting of acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, and p-toluene sulfonic acid. In a more preferred embodiment, the pharmaceutically acceptable salt of oxybutynin is oxybutynin chloride.

In one embodiment, the therapeutically effective amount of oxybutynin or a pharmaceutically acceptable salt thereof is a unit dosage form (single or divided dosage form as is known to one of skill in the art) in a range from about 0.1 mg to about 1 g administered q.d. Preferably, for administration orally, the therapeutically effective amount of oxybutynin or a pharmaceutically acceptable salt thereof is a unit dosage form in a range from about 1 mg to about 1 g administered q.d., more preferably, in a range from about 25 mg to about 700 mg administered q.d. Preferably, for administration by oral or intranasal inhalation, the therapeutically effective amount of oxybutynin or a pharmaceutically acceptable salt thereof is a unit dosage form in a range from about 0.1 mg to about 100 mg administered q.d. Yet more preferably, the therapeutically effective amount of oxybutynin or a pharmaceutically acceptable salt thereof is a unit dosage form in a range from about 1.25 mg to about 30 mg administered q.d (e.g., about 1.25 mg, about 2.5 mg, about 5 mg, about 10 mg, about 15 mg, about 20 mg, about 25 mg, about 30 mg administered q.d.). Still more preferably, the therapeutically effective amount of oxybutynin or a pharmaceutically acceptable salt thereof is a unit dosage form which is about 1.25 mg, about 2.5 mg, about 3 mg, about 3.5 mg, about 4 mg, about 4.5 mg, or about 5 mg administered b.i.d., t.i.d., or q.i.d. Yet still more preferably, the therapeutically effective amount of oxybutynin or a pharmaceutically acceptable salt thereof is a unit dosage form which is about 2.5 mg or about 5 mg administered b.i.d.

In one embodiment, the therapeutically effective amount of desloratadine or a pharmaceutically acceptable salt thereof is a unit dosage form (single or divided dosage form as is known to one of skill in the art) in a range from about 1.25 mg to about 45 mg administered q.d. (e.g., about 2 mg, about 4 mg, about 6 mg, about 8 mg, about 10 mg, about 12 mg, about 14 mg, about 16 mg, about 18 mg, about 20 mg, about 22 mg, about 24 mg, about 26 mg, about 28 mg, about 30 mg, about 32 mg, about 34 mg, about 36 mg, about 38 mg, about 40 mg, about 42 mg, about 44 mg administered q.d.). Preferably, the therapeutically effective amount of desloratadine or a pharmaceutically acceptable salt thereof is a unit dosage form in a range from about 1.25 mg to about 20 mg administered q.d. More preferably, the therapeutically effective amount of desloratadine or a pharmaceutically acceptable salt thereof is a unit dosage form in a range from about 5 mg to about 10 mg administered q.d. In one embodiment, the therapeutically effective amount of desloratadine or a pharmaceutically acceptable salt thereof is a unit dosage form which is about 1.25 mg, about 2.5 mg, about 5.0 mg, about 7.5 mg, about 10.0 mg, about 12.5 mg, about 15 mg, 17.5 mg, or about 20.0 mg administered q.d.

In one embodiment, the method comprising administering a therapeutically effective amount of darifenacin or oxybutynin, or a pharmaceutically acceptable salt thereof in combination with a therapeutically effective amount of desloratadine or a pharmaceutically acceptable salt thereof further comprises administering one or more additional pharmaceutically active agents. Preferably, one or more additional pharmaceutically active agents is another antihistamine; a decongestant; a corticosteroid; an expectorant; a composition to relieve oropharyngeal discomfort; a P2Y₂ receptor antagonist; a non-steroidal anti-inflammatory agent; a leukotriene antagonist; a syk kinase inhibitor; or a 5-lipoxygenase inhibitor.

In addition, the present invention provides methods for treating a skin disorder in a patient suffering therefrom comprising administering a therapeutically effective amount of a selective muscarinic acetylcholine receptor subtype M1 and/or M3 antagonist in combination with one or more pharmaceutically active agents.

In a preferred embodiment, the selective muscarinic acetylcholine receptor subtype M1 and/or M3 antagonist is glycopyrronium bromide, telenzepine, pirenzepine, oxybutynin, desethyloxybutynin, himbacine, AQ-RA 741, darifenacin, hexahydrosila-difenidol, p-flurohexahydro-sila-difenidol (p-F-HHSiD)), 4-diphenylacetoxy-N-methylpiperidine (4-DAMP) methiodide, or 4-DAMP methobromide, or a pharmaceutically acceptable salt of any of these agents, or a combination of one or more of these agents.

In one embodiment, the selective muscarinic acetylcholine receptor subtype M1 and/or M3 antagonist is a selective muscarinic acetylcholine receptor subtype M1 antagonist. Preferably, the selective muscarinic acetylcholine receptor subtype M1 antagonist is glycopyrronium bromide, telenzepine, or pirenzepine, or a pharmaceutically acceptable salt of any of these agents, or a combination of one or more of these agents. More preferably, the selective muscarinic acetylcholine receptor subtype Ml antagonist is pirenzepine or a pharmaceutically acceptable salt thereof. Preferably, the unit dosage form (single or divided dosage form as is known to one of skill in the art) of pirenzepine is in the range of about 2.5 mg to about 250 mg. More preferably, the unit dosage form of pirenzepine is in the range of about 100 mg to about 150 mg. Still more preferably, the unit dosage form of pirenzepine is about 50 mg administered b.i.d or t.i.d.

In another embodiment, the selective muscarinic acetylcholine receptor subtype M1 and/or M3 antagonist is a selective muscarinic acetylcholine receptor subtype M3 antagonist. Preferably, the selective muscarinic acetylcholine receptor subtype M3 antagonist is himbacine, AQ-RA 741, darifenacin, hexahydrosila-difenidol, p-flurohexahydro-sila-difenidol (p-F-HHSiD)), 4-diphenylacetoxy-N-methylpiperidine (4-DAMP) methiodide, or 4-DAMP methobromide, or a pharmaceutically acceptable salt of any of these agents, or a combination of one or more of these agents. More preferably, the selective muscarinic acetylcholine receptor subtype M3 antagonist is darifenacin or a pharmaceutically acceptable salt thereof.

In another embodiment, the selective muscarinic acetylcholine receptor subtype M1 and/or M3 antagonist is a selective muscarinic acetylcholine receptor subtype M1 and M3 antagonist. Preferably, the selective muscarinic acetylcholine receptor subtype M1 and M3 antagonist is oxybutynin, desethyloxybutynin, or a pharmaceutically acceptable salt of any of these agents, or a combination of one or more of these agents. More preferably, the selective muscarinic acetylcholine receptor subtype M1 and M3 antagonist is oxybutynin, desethyloxybutynin, or a pharmaceutically acceptable salt of any of these agents.

In yet another embodiment, one or more pharmaceutically active agents is an antihistamine. Preferably, the antihistamine is azatadine, azelastine, acrivastine, brompheniramine, cetirizine, chlorpheniramine, clemastine, cyclizine, carebastine, cyproheptadine, carbinoxamine, desloratadine, doxylamine, dimethindene, ebastine, epinastine, efletirizine, fexofenadine, hydroxyzine, ketotifen, loratadine, levocabastine, levocetirizine, mizolastine, mequitazine, mianserin, noberastine, meclizine, norastemizole, picumast, pyrilamine, promethazine, tripelennamine, temelastine, trimeprazine, triprolidine, thioperamide, impromidine, burimamide, clobenpropit, impentamine, mifetidine, S-sopromidine, R-sopromidine, 3-(imidazol-4-yl)-propylguanidine (SKF-91486), 3-[(4-chlorophenyl)methyl]-5-[2-(1H-imidazol-4yl)ethyl] 1,2,3-oxadiazole (GR-175737), 4-(1-cyclohexylpentanoyl-4-piperidyl) 1H-imidazole (GT-2016), 2-{[2-[4(5)-imidazolyl]ethyl]thio}-5-nitropyridine (UCL-1199), or clozapine, or a pharmaceutically acceptable salt of any of these agents, or a combination of one or more of these agents. More preferably, the antihistamine is desloratadine or a pharmaceutically acceptable salt thereof.

In still yet another embodiment, one or more pharmaceutically active agents is a decongestant. Preferably, the decongestant is a histamine H₃ receptor antagonist (e.g., thioperamide, impromidine, burimamide, clobenpropit, impentamine, mifetidine, S-sopromidine, R-sopromidine, 3-(imidazol-4-yl)-propylguanidine (SKF-91486), 3-[(4-chlorophenyl)methyl]-5-[2-(1H-imidazol-4yl)ethyl] 1,2,3-oxadiazole (GR-175737), 4-(1-cyclohexylpentanoyl-4-piperidyl) 1H-imidazole (GT-2016), 2-{[2-[4(5)-imidazolyl]ethyl]thio}-5-nitropyridine (UCL-1199), clozapine), levmetamfetamine, ephedrine, ephedrine hydrochloride, ephedrine sulfate, naphazoline hydrochloride, oxymetazoline hydrochloride, phenylephrine hydrochloride, propylhexedrine, xylometazoline hydrochloride, phenylpropanolamine, phenylephrine, or pseudoephedrine, or a pharmaceutically acceptable salt of any of these agents, or a combination of one or more of these agents. More preferably, the decongestant is phenylephrine or pseudoephedrine, or a pharmaceutically acceptable salt of any of these agents.

In yet other embodiments, one or more pharmaceutically active agents is a corticosteroid; an expectorant; a composition to relieve oropharyngeal discomfort; a P2Y₂ receptor antagonist; a non-steroidal anti-inflammatory agent; a leukotriene antagonist; a syk kinase inhibitor; or a 5-lipoxygenase inhibitor.

In one preferred embodiment, the method for treating a skin disorder in a patient suffering therefrom comprises administering a therapeutically effective amount of darifenacin or a pharmaceutically acceptable salt thereof in combination with a therapeutically effective amount of desloratadine or a pharmaceutically acceptable salt thereof.

In another preferred embodiment, the method for treating a skin disorder in a patient suffering therefrom comprises administering a therapeutically effective amount of oxybutynin or a pharmaceutically acceptable salt thereof in combination with a therapeutically effective amount of desloratadine or a pharmaceutically acceptable salt thereof.

In one embodiment, the pharmaceutically acceptable salt of oxybutynin is prepared from a pharmaceutically acceptable acid addition salt selected from the group consisting of acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, and p-toluene sulfonic acid. In a more preferred embodiment, the pharmaceutically acceptable salt of oxybutynin is oxybutynin chloride.

In one embodiment, the therapeutically effective amount of oxybutynin or a pharmaceutically acceptable salt thereof is a unit dosage form (single or divided dosage form as is known to one of skill in the art) in a range from about 0.1 mg to about 1 g administered q.d. Preferably, for administration orally, the therapeutically effective amount of oxybutynin or a pharmaceutically acceptable salt thereof is a unit dosage form in a range from about 1 mg to about 1 g administered q.d., more preferably, in a range from about 25 mg to about 700 mg administered q.d. Preferably, for administration by oral or intranasal inhalation, the therapeutically effective amount of oxybutynin or a pharmaceutically acceptable salt thereof is a unit dosage form in a range from about 0.1 mg to about 100 mg administered q.d. Yet more preferably, the therapeutically effective amount of oxybutynin or a pharmaceutically acceptable salt thereof is a unit dosage form in a range from about 1.25 mg to about 30 mg administered q.d (e.g., about 1.25 mg, about 2.5 mg, about 5 mg, about 10 mg, about 15 mg, about 20 mg, about 25 mg, about 30 mg administered q.d.). Still more preferably, the therapeutically effective amount of oxybutynin or a pharmaceutically acceptable salt thereof is a unit dosage form which is about 1.25 mg, about 2.5 mg, about 3 mg, about 3.5 mg, about 4 mg, about 4.5 mg, or about 5 mg administered b.i.d., t.i.d., or q.i.d. Yet still more preferably, the therapeutically effective amount of oxybutynin or a pharmaceutically acceptable salt thereof is a unit dosage form which is about 2.5 mg or about 5 mg administered b.i.d.

In one embodiment, the therapeutically effective amount of desloratadine or a pharmaceutically acceptable salt thereof is a unit dosage form (single or divided dosage form as is known to one of skill in the art) in a range from about 1.25 mg to about 45 mg administered q.d. (e.g., about 2 mg, about 4 mg, about 6 mg, about 8 mg, about 10 mg, about 12 mg, about 14 mg, about 16 mg, about 18 mg, about 20 mg, about 22 mg, about 24 mg, about 26 mg, about 28 mg, about 30 mg, about 32 mg, about 34 mg, about 36 mg, about 38 mg, about 40 mg, about 42 mg, about 44 mg administered q.d.). Preferably, the therapeutically effective amount of desloratadine or a pharmaceutically acceptable salt thereof is a unit dosage form in a range from about 1.25 mg to about 20 mg administered q.d. More preferably, the therapeutically effective amount of desloratadine or a pharmaceutically acceptable salt thereof is a unit dosage form in a range from about 5 mg to about 10 mg administered q.d. In one embodiment, the therapeutically effective amount of desloratadine or a pharmaceutically acceptable salt thereof is a unit dosage form which is about 1.25 mg, about 2.5 mg, about 5.0 mg, about 7.5 mg, about 10.0 mg, about 12.5 mg, about 15 mg, 17.5 mg, or about 20.0 mg administered q.d.

In one embodiment, the method comprising administering a therapeutically effective amount of darifenacin or oxybutynin, or a pharmaceutically acceptable salt thereof in combination with a therapeutically effective amount of desloratadine or a pharmaceutically acceptable salt thereof further comprises administering one or more additional pharmaceutically active agents. Preferably, one or more additional pharmaceutically active agents is another antihistamine; a decongestant; a corticosteroid; an expectorant; a composition to relieve oropharyngeal discomfort; a P2Y₂ receptor antagonist; a non-steroidal anti-inflammatory agent; a leukotriene antagonist; a syk kinase inhibitor; or a 5-lipoxygenase inhibitor.

In addition, the present invention provides methods for treating allergic conjunctivitis in a patient suffering therefrom comprising administering a therapeutically effective amount of a selective muscarinic acetylcholine receptor subtype M1 and/or M3 antagonist in combination with one or more pharmaceutically active agents.

In a preferred embodiment, the selective muscarinic acetylcholine receptor subtype M1 and/or M3 antagonist is glycopyrronium bromide, telenzepine, pirenzepine, oxybutynin, desethyloxybutynin, himbacine, AQ-RA 741, darifenacin, hexahydrosila-difenidol, p-flurohexahydro-sila-difenidol (p-F-HHSiD)), 4-diphenylacetoxy-N-methylpiperidine (4-DAMP) methiodide, or 4-DAMP methobromide, or a pharmaceutically acceptable salt of any of these agents, or a combination of one or more of these agents.

In one embodiment, the selective muscarinic acetylcholine receptor subtype M1 and/or M3 antagonist is a selective muscarinic acetylcholine receptor subtype M1 antagonist. Preferably, the selective muscarinic acetylcholine receptor subtype M1 antagonist is glycopyrronium bromide, telenzepine, or pirenzepine, or a pharmaceutically acceptable salt of any of these agents, or a combination of one or more of these agents. More preferably, the selective muscarinic acetylcholine receptor subtype M1 antagonist is pirenzepine or a pharmaceutically acceptable salt thereof. Preferably, the unit dosage form (single or divided dosage form as is known to one of skill in the art) of pirenzepine is in the range of about 2.5 mg to about 250 mg. More preferably, the unit dosage form of pirenzepine is in the range of about 100 mg to about 150 mg. Still more preferably, the unit dosage form of pirenzepine is about 50 mg administered b.i.d or t.i.d.

In another embodiment, the selective muscarinic acetylcholine receptor subtype M1 and/or M3 antagonist is a selective muscarinic acetylcholine receptor subtype M3 antagonist. Preferably, the selective muscarinic acetylcholine receptor subtype M3 antagonist is himbacine, AQ-RA 741, darifenacin, hexahydrosila-difenidol, p-flurohexahydro-sila-difenidol (p-F-HHSiD)), 4-diphenylacetoxy-N-methylpiperidine (4-DAMP) methiodide, or 4-DAMP methobromide, or a pharmaceutically acceptable salt of any of these agents, or a combination of one or more of these agents. More preferably, the selective muscarinic acetylcholine receptor subtype M3 antagonist is darifenacin or a pharmaceutically acceptable salt thereof.

In another embodiment, the selective muscarinic acetylcholine receptor subtype M1 and/or M3 antagonist is a selective muscarinic acetylcholine receptor subtype M1 and M3 antagonist. Preferably, the selective muscarinic acetylcholine receptor subtype M1 and M3 antagonist is oxybutynin, desethyloxybutynin, or a pharmaceutically acceptable salt of any of these agents, or a combination of one or more of these agents. More preferably, the selective muscarinic acetylcholine receptor subtype M1 and M3 antagonist is oxybutynin, desethyloxybutynin, or a pharmaceutically acceptable salt of any of these agents.

In yet another embodiment, one or more pharmaceutically active agents is an antihistamine. Preferably, the antihistamine is azatadine, azelastine, acrivastine, brompheniramine, cetirizine, chlorpheniramine, clemastine, cyclizine, carebastine, cyproheptadine, carbinoxamine, desloratadine, doxylamine, dimethindene, ebastine, epinastine, efletirizine, fexofenadine, hydroxyzine, ketotifen, loratadine, levocabastine, levocetirizine, mizolastine, mequitazine, mianserin, noberastine, meclizine, norastemizole, picumast, pyrilamine, promethazine, tripelennamine, temelastine, trimeprazine, triprolidine, thioperamide, impromidine, burimamide, clobenpropit, impentamine, mifetidine, S-sopromidine, R-sopromidine, 3-(imidazol-4-yl)-propylguanidine (SKF-91486), 3-[(4-chlorophenyl)methyl]-5-[2-(1H-imidazol-4yl)ethyl] 1,2,3-oxadiazole (GR-175737), 4-(1-cyclohexylpentanoyl-4-piperidyl) 1H-imidazole (GT-2016), 2-{[2-[4(5)-imidazolyl]ethyl]thio}-5-nitropyridine (UCL-1199), or clozapine, or a pharmaceutically acceptable salt of any of these agents, or a combination of one or more of these agents. More preferably, the antihistamine is desloratadine or a pharmaceutically acceptable salt thereof.

In still yet another embodiment, one or more pharmaceutically active agents is a decongestant. Preferably, the decongestant is a histamine H₃ receptor antagonist (e.g., thioperamide, impromidine, burimamide, clobenpropit, impentamine, mifetidine, S-sopromidine, R-sopromidine, 3-(imidazol-4-yl)-propylguanidine (SKF-91486), 3-[(4-chlorophenyl)methyl]-5-[2-(1H-imidazol-4yl)ethyl] 1,2,3-oxadiazole (GR-175737), 4-(1-cyclohexylpentanoyl-4-piperidyl) 1H-imidazole (GT-2016), 2-{[2-[4(5)-imidazolyl]ethyl]thio}-5-nitropyridine (UCL-1199), clozapine), levmetamfetamine, ephedrine, ephedrine hydrochloride, ephedrine sulfate, naphazoline hydrochloride, oxymetazoline hydrochloride, phenylephrine hydrochloride, propylhexedrine, xylometazoline hydrochloride, phenylpropanolamine, phenylephrine, or pseudoephedrine, or a pharmaceutically acceptable salt of any of these agents, or a combination of one or more of these agents. More preferably, the decongestant is phenylephrine or pseudoephedrine, or a pharmaceutically acceptable salt of any of these agents.

In yet other embodiments, one or more pharmaceutically active agents is a corticosteroid; an expectorant; a composition to relieve oropharyngeal discomfort; a P2Y₂ receptor antagonist; a non-steroidal anti-inflammatory agent; a leukotriene antagonist; a syk kinase inhibitor; or a 5-lipoxygenase inhibitor.

In one preferred embodiment, the method for treating allergic conjunctivitis in a patient suffering therefrom comprises administering a therapeutically effective amount of darifenacin or a pharmaceutically acceptable salt thereof in combination with a therapeutically effective amount of desloratadine or a pharmaceutically acceptable salt thereof.

In another preferred embodiment, the method for treating allergic conjunctivitis in a patient suffering therefrom comprises administering a therapeutically effective amount of oxybutynin or a pharmaceutically acceptable salt thereof in combination with a therapeutically effective amount of desloratadine or a pharmaceutically acceptable salt thereof.

In one embodiment, the pharmaceutically acceptable salt of oxybutynin is prepared from a pharmaceutically acceptable acid addition salt selected from the group consisting of acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, and p-toluene sulfonic acid. In a more preferred embodiment, the pharmaceutically acceptable salt of oxybutynin is oxybutynin chloride.

In one embodiment, the therapeutically effective amount of oxybutynin or a pharmaceutically acceptable salt thereof is a unit dosage form (single or divided dosage form as is known to one of skill in the art) in a range from about 0.1 mg to about 1 g administered q.d. Preferably, for administration orally, the therapeutically effective amount of oxybutynin or a pharmaceutically acceptable salt thereof is a unit dosage form in a range from about 1 mg to about 1 g administered q.d., more preferably, in a range from about 25 mg to about 700 mg administered q.d. Preferably, for administration by oral or intranasal inhalation, the therapeutically effective amount of oxybutynin or a pharmaceutically acceptable salt thereof is a unit dosage form in a range from about 0.1 mg to about 100 mg administered q.d. Yet more preferably, the therapeutically effective amount of oxybutynin or a pharmaceutically acceptable salt thereof is a unit dosage form in a range from about 1.25 mg to about 30 mg administered q.d (e.g., about 1.25 mg, about 2.5 mg, about 5 mg, about 10 mg, about 15 mg, about 20 mg, about 25 mg, about 30 mg administered q.d.). Still more preferably, the therapeutically effective amount of oxybutynin or a pharmaceutically acceptable salt thereof is a unit dosage form which is about 1.25 mg, about 2.5 mg, about 3 mg, about 3.5 mg, about 4 mg, about 4.5 mg, or about 5 mg administered b.i.d., t.i.d., or q.i.d. Yet still more preferably, the therapeutically effective amount of oxybutynin or a pharmaceutically acceptable salt thereof is a unit dosage form which is about 2.5 mg or about 5 mg administered b.i.d.

In one embodiment, the therapeutically effective amount of desloratadine or a pharmaceutically acceptable salt thereof is a unit dosage form (single or divided dosage form as is known to one of skill in the art) in a range from about 1.25 mg to about 45 mg administered q.d. (e.g., about 2 mg, about 4 mg, about 6 mg, about 8 mg, about 10 mg, about 12 mg, about 14 mg, about 16 mg, about 18 mg, about 20 mg, about 22 mg, about 24 mg, about 26 mg, about 28 mg, about 30 mg, about 32 mg, about 34 mg, about 36 mg, about 38 mg, about 40 mg, about 42 mg, about 44 mg administered q.d.). Preferably, the therapeutically effective amount of desloratadine or a pharmaceutically acceptable salt thereof is a unit dosage form in a range from about 1.25 mg to about 20 mg administered q.d. More preferably, the therapeutically effective amount of desloratadine or a pharmaceutically acceptable salt thereof is a unit dosage form in a range from about 5 mg to about 10 mg administered q.d. In one embodiment, the therapeutically effective amount of desloratadine or a pharmaceutically acceptable salt thereof is a unit dosage form which is about 1.25 mg, about 2.5 mg, about 5.0 mg, about 7.5 mg, about 10.0 mg, about 12.5 mg, about 15 mg, 17.5 mg, or about 20.0 mg administered q.d.

In one embodiment, the method comprising administering a therapeutically effective amount of darifenacin or oxybutynin, or a pharmaceutically acceptable salt thereof in combination with a therapeutically effective amount of desloratadine or a pharmaceutically acceptable salt thereof further comprises administering one or more additional pharmaceutically active agents. Preferably, one or more additional pharmaceutically active agents is another antihistamine; a decongestant; a corticosteroid; an expectorant; a composition to relieve oropharyngeal discomfort; a P2Y₂ receptor antagonist; a non-steroidal anti-inflammatory agent; a leukotriene antagonist; a syk kinase inhibitor; or a 5-lipoxygenase inhibitor.

In a particular embodiment, the compositions of the present invention are useful for the treatment or alleviation of symptoms of anterior rhinitis.

### DETAILED DESCRIPTION OF THE INVENTION

As used herein, the following terms shall have the definitions set forth below.

As used herein, the phrase “respiratory disorder associated with the production of mucus glycoprotein” includes respiratory disorders that would be improved by inhibition of nasal secretion and the pathway triggering nasal secretion. For example, allergic rhinitis, non-allergic rhinitis (e.g., vasomotor rhinitis and non-allergic rhinitis eosinophilia syndrome (NARES)), infectious rhinitis (e.g., symptoms associated with the common cold), sinusitits (i.e., an inflammation, or swelling, of the mucous membranes that line the sinus cavities), and post-nasal drip (i.e., wherein drainage from the nose and sinuses drips down the back of the throat). In addition, respiratory disorders associated with the production of mucus glycoprotein include conditions that are triggered or exacerbated by nasal secretion. For example, respiratory congestion, cough, and allergic airway disease manifested by symptoms including wheezing, chest tightness, cough, and dyspnea.

As used herein, the phrase “skin disorder” includes the skin reactions of urticaria and angioedema. These skin disorders may be triggered by exposure to certain foods, medications, or virus infections. Urticarira (commonly referred to as hives or welts) are red, itchy, raised areas of the skin of varying shapes and sizes. Urticarira are the result of release of histamine and other compounds from mast cells that cause serum to leak from local blood vessels and thereby cause swelling in the skin. Angioedema is a form of tissue swelling similar to urticaria, but involving deeper skin tissues (i.e., “deep hives”) and generally lasting longer than urticaria.

As used herein, the phrase “allergic conjunctivitis” refers to an irritation by an allergen of the clear, thin membrane called the conjunctiva that covers the eyeball and the inside of the eyelids. Symptoms may include swollen eyes, itchy/burning eyes, tearing, and ocular redness. Some common allergens include pollen from trees, grass and ragweed, animal skin and secretions such as saliva, perfumes and cosmetics, skin medicines, air pollution and smoke.

As used herein, the phrase “selective muscarinic acetylcholine receptor subtype M1 and/or M3 antagonist” means an agent that inhibits cell signaling from muscarinic acetylcholine receptor subtype M1 and/or M3 with greater potency than muscarinic acetylcholine receptor subtype M2. In one embodiment, the agent inhibits cell signaling from muscarinic acetylcholine receptor subtype M1 and/or M3 with about 5-fold greater potency than muscarinic acetylcholine receptor subtype M2. In another embodiment, the agent inhibits cell signaling from muscarinic acetylcholine receptor subtype M1 and/or M3 with about 7.5-fold greater potency than muscarinic acetylcholine receptor subtype M2. In yet another embodiment, the agent inhibits cell signaling from muscarinic acetylcholine receptor subtype M1 and/or M3 with about 10-fold greater potency than muscarinic acetylcholine receptor subtype M2.

As used herein, the phrase “therapeutically effective amount” with respect to a selective muscarinic acetylcholine receptor subtype M1 and/or M3 antagonist means a therapeutic benefit in the treatment or management of the referenced disorder (e.g., a respiratory disorder associated with the production of mucus glycoprotein, a skin disorder, or allergic conjunctivitis).

As used herein, the phrase “therapeutically effective amount” with respect to one or more pharmaceutically active agents means a therapeutic benefit in the treatment or management of the referenced disorder (e.g., a respiratory disorder associated with the production of mucus glycoprotein, a skin disorder, or allergic conjunctivitis).

As used herein, the phrase “pharmaceutically active agent” refers to an antihistamine, a decongestant, a corticosteroid, an expectorant, a composition to relieve oropharyngeal discomfort, a P2Y₂ receptor antagonist, a non-steroidal anti-inflammatory agent, a leukotriene antagonist, a syk kinase inhibitor, or a 5-lipoxygenase inhibitor.

As used herein the phrase “pharmaceutically acceptable salt” refers to a non-toxic salt prepared from pharmaceutically acceptable acids or bases (including inorganic acids or bases, or organic acids or bases). Examples of such inorganic acids are hydrochloric, hydrobromic, hydroiodic, sulfuric, and phosphoric. Appropriate organic acids may be selected, for example, from aliphatic, aromatic, carboxylic and sulfonic classes of organic acids, examples of which are formic, acetic, propionic, succinic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, algenic, and galacturonic. Examples of such inorganic bases include metallic salts made from aluminum, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (N-methylgulcaine), lysine, and procaine. Examples of suitable pharmaceutically acceptable acid addition salts for oxybutynin include acetic, benzenesulfonic (besylate), benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, and p-toluene sulfonic. The hydrochloride has particular utility.

As used herein, the phrase “medicinal agent” includes a substance that is a selective muscarinic acetylcholine receptor subtype M1 and/or M3 antagonist, and/or a pharmaceutically active agent.

As used herein the phrase “dosage form” refers to a composition comprising at least one medicinal agent and a carrier formulated into a delivery system (i.e., tablet, capsule, oral gel, orally consumable films, orally disintegrating tablet (e.g., Reditabs®), syrup, nasal spray, powders for constitution or suspension, powder (sprinkles) that is put onto foods) in association with at least one inactive ingredient.

As used herein the term “capsule” refers to a special container or enclosure made of methyl cellulose, polyvinyl alcohols, or denatured gelatins or starch for holding or containing a composition comprising a formulation of the present invention and a carrier. Hard shell capsules are typically made of blends of relatively high gel strength bone and pork skin gelatins. The capsule itself may contain small amounts of dyes, opaquing agents, plasticizers, and preservatives.

As used herein the term “tablet” refers to a compressed or molded solid dosage form containing a composition comprising a formulation of the present invention and a carrier with suitable diluents. The tablet can be prepared by compression of mixtures or granulations obtained by wet granulation, dry granulation or by compaction.

As used herein the phrase “oral gel” refers to a composition comprising a formulation of the present invention and a carrier dispersed or solubilized in a hydrophilic semi-solid matrix.

As used herein the phrase “orally consumable film” refers to a composition comprising a formulation of the present invention and an edible film carrier.

As used herein the phrase “powders for constitution” refers to powder blends containing a composition comprising a formulation of the present invention and a carrier with suitable diluents which can be suspended in water or juices.

As used herein the term “diluent” refers to a substance that usually makes up the major portion of the composition or dosage form. Suitable diluents include sugars such as lactose, sucrose, mannitol, and sorbitol; starches derived from wheat, corn rice, and potato; and celluloses such as microcrystalline cellulose. The amount of diluent in the composition can range from about 10% to about 90% by weight of the total composition, preferably from about 25% to about 75%, more preferably from about 30% to about 60% by weight, even more preferably from about 12% to about 60%.

As used herein the term “disintegrant” refers to a substance added to the dosage form to help it break apart (disintegrate) and release the medicinal agent(s). Suitable disintegrants include starches; “cold water soluble” modified starches such as sodium carboxymethyl starch; natural and synthetic gums such as locust bean, karaya, guar, tragacanth, and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescent mixtures. The amount of disintegrant in the composition can range from about 2% to about 15% by weight of the composition, more preferably from about 4% to about 10% by weight.

As used herein the term “binder” refers to a substance that binds or “glues” powders together and makes them cohesive by forming granules, thus serving as the “adhesive” in the dosage form. Binders add cohesive strength already available in the diluent or bulking agent. Suitable binders include sugars such as sucrose; starches derived from wheat, corn rice, and potato; natural gums such as acacia, gelatin, and tragacanth; derivatives of seaweed such as alginic acid, sodium alginate, and ammonium calcium alginate; cellulosic materials such as methylcellulose, sodium carboxymethylcellulose, and hydroxypropylmethylcellulose; polyvinylpyrrolidinone; and inorganics such as magnesium aluminum silicate. The amount of binder in the composition can range from about 2% to about 20% by weight of the composition, more preferably from about 3% to about 10% by weight, even more preferably from about 3% to about 6% by weight.

As used herein the term “lubricant” refers to a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols, and d'l-leucine. Lubricants are usually added at the very last step before compression, since they must be present on the surfaces of the granules and in between them and the parts of the tablet press. The amount of lubricant in the composition can range from about 0.2% to about 5% by weight of the composition, preferably from about 0.5% to about 2%, more preferably from about 0.3% to about 1.5% by weight.

As used herein the term “glidant” refers to a substance that prevents caking and improves the flow characteristics of granulations, so that flow is smooth and uniform. Suitable glidants include silicon dioxide and talc. The amount of glidant in the composition can range from about 0.1% to about 5% by weight of the total composition, preferably from about 0.5% to about 2% by weight.

As used herein the phrase “coloring agent” refers to a substance that provides coloration to the composition or the dosage form. Such substances can include food grade dyes and food grade dyes adsorbed onto a suitable adsorbent such as clay or aluminum oxide. The amount of the coloring agent can vary from about 0.1% to about 5% by weight of the composition, preferably from about 0.1% to about 1%.

As used herein the term “bioavailability” refers to the rate and extent to which the medicinal agent is absorbed into the systemic circulation from an administered dosage form as compared to a standard or control, as well as to topical bioavailability.

One preferred selective muscarinic acetylcholine receptor subtype M1 and M3 antagonist is oxybutynin or a pharmaceutically acceptable salt thereof (e.g., oxybutynin chloride). A racemic mixture of oxybutynin chloride is available under the trademark Ditropan® and Ditropan XL® by Ortho-McNeil Pharmaceutical, Inc., Raritan, N.J. A racemic mixture of R- and S-enantiomers of oxybutynin or a pharmaceutically acceptable salt thereof may be used in the formulations and methods of the present invention. Alternatively, the R-enantiomer or the S-enantiomer, substantially free of the S-enantiomer or the R-enantiomer, respectively, may be used in the formulations and methods of the present invention. Use of the S-enantiomer of oxybutynin for the treatment of asthma is disclosed in U.S. Pat. No. 6,294,582. Likewise, the selective muscarinic acetylcholine receptor subtype M1 and M3 antagonist may be a derivative of oxybutynin (e.g., desethyloxybutynin), or a pharmaceutically acceptable salt thereof. A racemic mixture of R- and S-enantiomers of desethyloxybutynin may be used in the formulations and methods of the present invention. Alternatively, the R-enantiomer or the S-enantiomer of desethyloxybutynin, substantially free of the S-enantiomer or the R-enantiomer, respectively, may be used in the formulations and methods of the present invention. Quaternary ammonium derivatives of oxybutynin and methods of their use for the treatment of asthma, chronic obstructive pulmonary disease, allergic rhinitis, or infectious rhinitis are disclosed in WO 04/039763.

The present invention also provides an antihistamine in combination with a selective muscarinic acetylcholine receptor subtype M1 and/or M3 antagonist. One preferred antihistamine for use with a selective muscarinic acetylcholine receptor subtype M1 and/or M3 antagonist is desloratadine, available under the trademark Clarinex® by Schering Corporation, Kenilworth, N.J. This compound is described in Quercia et al., Hosp Formul, 28:137-53 (1993), in U.S. Pat. 4,659,716, and in WO 96/20708. The use of desloratadine for the treatment of congestion is disclosed in U.S. Pat. No. 6,432,972. Desloratadine is an antagonist of the H₁ histamine receptor protein. The H₁ receptors are those that mediate the response antagonized by conventional antihistamines. H₁ receptors are present, for example, in the ileum, the skin, and the bronchial smooth muscle of man and other mammals. The amount of desloratadine which can be employed in a unit dosage form (single or divided dosage form as is known to one of skill in the art) of the present compositions can range from about 1.25 mg to about 45 mg, also from about 1.25 mg to about 20 mg, also from about 5 to about 10 mg. Preferably, the dosage amount is 1.25 mg, 2.5 mg, 5.0 mg, 10.0 mg, or 20.0 mg. More preferably, the dosage amount is 5.0 mg.

Another antihistamine for use with a selective muscarinic acetylcholine receptor subtype M1 and/or M3 antagonist is loratadine, available under the trademark Claritin® from Schering Corporation, Kenilworth, N.J. Loratadine, described in U.S. Pat. No. 4,282,233, is a potent tricyclic and antihistaminic drug of slow release, with a selective antagonist of peripheric H₁ receptors activity.

Another antihistamine for use with a selective muscarinic acetylcholine receptor subtype M1 and/or M3 antagonist is fexofenadine, available under the trademark Allegra® from Aventis, Strasbourg, France. Fexofenadine is described in U.S. Pat. No. 5,578,610. Preferably, the pharmaceutically acceptable salt is fexofenadine hydrochloride. The amount of fexofenadine which can be employed in a unit dosage form (single or divided dosage form as is known to one of skill in the art) of the present composition can range from about 30 mg to 200 mg, also from about 30 mg to about 180 mg, (e.g., about 30 mg, about 60 mg, about 120 mg, about 180 mg).

Another antihistamine for use with a selective muscarinic acetylcholine receptor subtype M1 and/or M3 antagonist is cetirizine, available under the trademark ZYRTEC® from Pfizer Inc., New York, N.Y. Cetirizine hydrochloride is a racemic compound with an empirical formula of C₂₁H₂₅ClN₂O₃·2HCl. Cetirizine hydrochloride is a white, crystalline powder that is water-soluble. U.S. Pat. No. 6,258,814 discloses cetirizine as well as the use of cetirizine as a sleep aid. The amount of cetirizine which can be employed in a unit dosage form (single or divided dosage form as is known to one of skill in the art) of the present composition can range from about 1 mg to 40 mg, also from about 2.5 mg to about 10 mg (e.g., about 5 mg). The levo isomer of cetirizine may also be combined with a selective muscarinic acetylcholine receptor subtype M1 and/or M3 antagonist in the formulations of the present invention. U.S. Pat. No. 6,319,927 discloses a method of using levocetirizine as a sleep aid.

In addition, other antihistamines useful for formulations of the present invention include azatadine, azelastine, acrivastine, brompheniramine, chlorpheniramine, clemastine, cyclizine, carebastine, cyproheptadine, carbinoxamine, doxylamine, dimethindene, ebastine, epinastine, efletirizine, hydroxyzine, ketotifen, levocabastine, mizolastine, mequitazine, mianserin, noberastine, meclizine, norastemizole, picumast, pyrilamine, promethazine, tripelennamine, temelastine, trimeprazine, and triprolidineastemizole. Preferably, the amount of norastemizole which can be employed in a unit dosage form (single or divided dosage form as is known to one of skill in the art) of the present composition is about 10 mg or more.

The present invention provides a decongestant in combination with a selective muscarinic acetylcholine receptor subtype M1 and/or M3 antagonist. Preferably, the decongestant is an oral or nasal decongestant. Examples of nasal decongestants useful in the present invention include the sympathomimetic amine nasal decongestants. Such nasal decongestants approved for use in the United States include levmetamfetamine (also known as 1-desoxyephedrine), ephedrine, ephedrine hydrochloride, ephedrine sulfate, naphazoline hydrochloride, oxymetazoline hydrochloride, phenylephrine hydrochloride, propylhexedrine, and xylometazoline hydrochloride. Oral decongestants for use in the present invention include phenylpropanolamine, phenylephrine, and pseudoephedrine. Pseudoephedrine as well as pharmaceutically acceptable acid additional salts (e.g., those of HCl or H₂SO₄), is a sympathomimetic drug recognized by those skilled in the art as a safe therapeutic agent effective for treating nasal congestion and is commonly administered orally and concomitantly with an antihistamine for treatment of nasal congestion associated with allergic rhinitis. The use of pseudoephedrine as a nasal decongestant in the present invention is preferred in amounts of about 120 mg pseudoephedrine sulfate dosed one to 4 times daily. However, lesser amounts of pseudoephedrine sulfate may be used in combination with a selective muscarinic acetylcholine receptor subtype M1 and/or M3 antagonist.

The present invention also provides a corticosteroid in combination with a selective muscarinic acetylcholine receptor subtype M1 and/or M3 antagonist. Corticosteroids for use in the present invention include dexamethasone, butoxicort, rofleponide, budesonide, deflazacort, ciclesonide, fluticasone, beclomethasone, loteprednol, mometasone, betametasone, or triamcinolone. For instance, when the corticosteroid is fluticasone, it may be administered at the dose of 2 sprays of 50 μg of fluticasone propionate each in each nostril once daily. Alternatively, it may be administered at a dose of fluticasone is 1 spray of 50 μg of fluticasone propionate each in each nostril once daily. When the corticosteroid is triamcinolone, it may be administered at a dose of triamcinolone is 220 μg per day as two sprays in each nostril once daily. Alternatively, it may be administered at a dose of 110 μg per day as one spray in each nostril once daily. When the corticosteroid is budesonide, the administered dose of budesonide may be 64 μg per day administered as one spray per nostril of 32 μg once daily.

A particularly preferred steroid is mometasone furoate. Mometasone furoate is a corticosteroid approved for topical dermatologic use to treat inflammatory and/or pruritic manifestations of corticosteroid-responsive dermatoses. The compound may be prepared in accordance with the procedures disclosed in U.S. Pat. Nos. 4,472,393, 4,731,447, 4,873,335, and 6,127,353, all of which are hereby incorporated by reference in their entirety. Mometasone furoate is a topically active steroid which is not readily bioavailable. It is commercially available as a spray for intra-nasal administration under the name of Nasonex®. Use of mometasone furoate for the treatment of airway passages and lung diseases is disclosed in U.S. Pat. Nos. 6,677,323, 6,677,322, 6,365,581, 6,187,765, 6,068,832, 6,057,307, 5,889,015, 5,837,699, and 5,474,759, all of which are incorporated by reference in their entirety. For the treatment of allergic, non-allergic rhinitis and/or inflammatory disorders of the upper or lower airway passages, the substantially non-systematically bioavailable amount of mometasone furoate which may be administered as an aqueous suspension or dry powder is in the range of about 10 μg/day to about 5000 μg/day, about 10 μg/day to about 4000 μg/day, about 10 μg/day to about 2000 μg/day, about 25 μg/day to about 1000 μg/day, about 25 μg/day to about 400 μg/day, about 25 μg/day to about 200 μg/day, about 25 μg/day to about 100 μg/day, or about 25 μg/day to about 50 μg/day in single or divided doses.

The present invention also provides an expectorant in combination with a selective muscarinic acetylcholine receptor subtype M1 and/or M3 antagonist. Ambroxol is a bromhexine metabolite, chemically identified as trans-4(2-amino-3,5-dibromobenzil, amine) ciclohexane hydrochloride, which has been widely used during more than two decades as an expectorant agent or stimulating pulmonary surfactant factor. The compound is described in U.S. Pat. No. 3,536,712. Guaiafenesin is an expectorant, whose technical name is 3-(2-methoxyphenoxy)-1,2-propanediol. The compound is described in U.S. Pat. No. 4,390,732. Terpin hydrate is an expectorant, whose technical name is 4-hydroxy-α, α, 4-trimethylcyclohexane-methanol. Potassium guaicolsulfonate is an expectorant, whose technical name is 3-hydroxy-4-methoxybenzenesulfonic acid mix with mono-potassium 4-hydroxy-3-methoxybenzenesulfonate. These combinations may be administered orally as set forth below.

The present invention also provides a composition to relieve oropharyngeal discomfort in combination with a selective muscarinic acetylcholine receptor subtype M1 and/or M3 antagonist. Compositions to relieve oropharyngeal discomfort, such as sore throat, cold or canker sores, painful gums, and other conditions include topical anesthetics such as phenol, hexylresorcinol, salicyl alcohol, benzyl alcohol, dyclonine, dibucaine, benzocaine, buticaine, cetylpyridinium chloride, diperidon, clove oil, menthol, camphor, eugenol, and others. Similarly, drugs that may be incorporated for application to the skin for relieving discomfort include lidocaine, benzocaine, tetracaine, dibucaine, pramoxine, diphenhydramine, benzyl alcohol, and others.

The present invention also provides a P2Y₂ receptor agonist in combination with a selective muscarinic acetylcholine receptor subtype M1 and/or M3 antagonist. Diquafosol tetrasodium is a P2Y₂ receptor agonist that activates receptors on the ocular surface and inner lining of the eyelid to stimulate the release of water, salt, mucin, and lipids—the key components of natural tears. Mucin is made in specialized cells and acts to lubricate surfaces. Lipids in the eye are oily substances that form the outer-most layer of the tear film and are responsible for the prevention of excess tear fluid evaporation. In preclinical testing, diquafosol reportedly increased the secretions of natural tear components. Diquafosol is available from Inspire Pharmaceuticals, Inc., Durham, NC. P2Y₂ receptor agonists are a new class of compounds being developed for the treatment of a variety of conditions, including chronic bronchitis and cystic fibrosis. Other mucolytic agents may include N-acetylcysteine and endogenous ligand compound UTP. These compositions may be administered either orally or nasally as set forth below in amounts that are known to one of skill in the art.

The present invention also provides a non-steroidal anti-inflammatory (“NSAID's”) agent in combination with a selective muscarinic acetylcholine receptor subtype M1 and/or M3 antagonist. Suitable NSAID's include acetylsalicylic acid, acetaminophen, indomethacin, diclofenac, piroxicam, tenoxicam, ibuprofen, naproxen, ketoprofen, nabumetone, ketorolac, azapropazone, mefenamic acid, tolfenamic acid, sulindac, diflunisal, tiaprofenic acid, podophyllotoxin derivatives, acemetacin, aceclofenac, droxicam, oxaprozin, floctafenine, phenylbutazone, proglumetacin, flurbiprofen, tolmetin, and fenbufen. These compositions may be administered either orally or nasally as set forth below in amounts that are known to one of skill in the art.

The present invention also provides a leukotriene antagonist in combination with a selective muscarinic acetylcholine receptor subtype M1 and/or M3 antagonist. Suitable leukotriene D₄ antagonists include zileuton, docebenone, piripost, ICI-D2318, MK-591, MK-886, sodium 1-(((R)-(3-(2-(6,7-difluoro-2-quinolinyl)ethynyl)phenyl) -3-(2-(2-hydroxy-2-propyl)phenyl)thio)methyl)cyclopropane-acetate; 1 -(((1(R)-(3-(2-(2,3-dichlorothieno[3,2-b]pyridin-5-yl)-(E)-ethenyl)phenyl )-3-(2-(1-hydroxy-1-methylethyl)phenyl)propyl)thio)-methyl)cyclopropaneacetic acid, praniukast, zafirlukast, and montelukast. These compositions may be administered either orally or nasally as set forth below in amounts that are known to one of skill in the art.

Montelukast is a leukotriene D₄ antagonist capable of antagonizing the receptors for the cysteinyl leukotrienes and is described in EP 0 480 717. A preferred pharmaceutically acceptable salt of montelukast is the monosodium salt, also known as montelukast sodium. The amount of montelukast which can be employed in a unit dosage form (single or divided dosage form as is known to one of skill in the art) of the present invention can range from about 1 mg to 100 mg, also from about 5 mg to about 20 mg, preferably about 10 mg.

The compound 1-(((R)-(3-(2-(6,7-difluoro-2-quinolinyl)ethenyl)phenyl)-3-(2-(2-hydroxy-2-propyl)phenyl)thio)methylcyclopropaneacetic acid is a leukotriene antagonist described in WO 97/28797 and U.S. Pat. No. 5,270,324. A pharmaceutically acceptable salt of this compound is the sodium salt, also known as sodium 1-(((R)-(3-(2-(6,7-difluoro-2-quinolinyl)ethenyl)phenyl)-3-(2-(2-hydroxy-2-propyl) phenyl)thio)-methylcyclopropaneacetate.

The compound 1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)-thio)methyl)cyclopropaneacetic acid is a leukotriene antagonist described in WO 97/28797 and U.S. Pat. No. 5,472,964. A pharmaceutically acceptable salt of this compound is the sodium salt, also known as sodium 1-(((1(R)-3(3-(2-(2,3-dichlorothieno[3,2-b] pyridin-5-yl )-(E)-ethenyl)phenyl)-3-(2-(1-hydroxy-1-methylethyl)phenyl)propyl)-thio)methyl)cyclopropaneacetate.

Pranlukast is a leukotriene antagonist described in WO 97/28797 and EP 0 173 516. The technical name for this compound is N-[4-oxo-2-(1 H-tetrazol-5-yl)-4H-1-benzopyran-8-yl]-p-(4-phenylbutoxy)benzamide. The amount of praniukast which can be employed in a unit dosage form (single or divided dosage form as is known to one of skill in the art) can range from about 100 mg to about 700 mg, preferably from about 112 mg to about 675 mg; also from about 225 mg to about 450 mg; also from about 225 mg to about 300 mg.

Zafirlukast is a leukotriene antagonist described in WO 97/28797 and EP 0 199 543. The technical name for this compound is cyclopentyl-3-[2-methoxy-4-[(o-tolylsulfonyl)carbamoyl] benzyl]-1-methylindole-5-carbamate.

The compound [2-[[2-(4-tert-butyl-2-thiazolyl)-5-benzofuranyl]oxymethyl]phenyl]acetic acid is a leukotriene antagonist and/or inhibitor whose method for preparation is described in U.S. Pat. No. 5,296,495 and Japanese Patent JP 08325265 A. An alternative name for this compound is 2-[[[2-[4-(1,1-dimethylethyl)-2-thiazolyl]-5-benzofuranyl]oxy]methyl]-benzeneacetic acid. The code number for this compound is FK011 or FR150011. The compound has a molecular formula of C₂₄H₂₃NO₄S and molecular weight of 421.52.

The present invention also provides a syk kinase inhibitor in combination with a selective muscarinic acetylcholine receptor subtype M1 and/or M3 antagonist. Syk kinase inhibitors include R112, available from Rigel Pharmaceuticals, Inc. A recent study reportedly showed a greater than 20% relative improvement for R112 over placebo (an absolute difference of 9% over placebo) and up to 38% improvement for R112 from baseline measurements (prior to drug initiation). In particular, symptoms most closely associated with chronic nasal congestion (e.g., stuffy nose) were reportedly improved with R112 over placebo.

The present invention also provides a 5-lipoxygenase inhibitor in combination with a selective muscarinic acetylcholine receptor subtype M1 and/or M3 antagonist. 5-lipoxygenase inhibitors include any agent, or compound that inhibits, restrains, retards or otherwise interacts with the enzymatic action of 5-lipoxygenase, such as, but not limited to, zileuton, docebenone, piripost, and the like. In addition, 5-lipoxygenase inhibitors include any agent or compound that inhibits, retrains, retards or otherwise interacts with the action or activity of 5-lipoxygenase activating protein, such as, but not limited to MK-591 and MK-886.

As will be evident to one of skill in the art, the formulations of the present invention may contain a selective muscarinic acetylcholine receptor subtype M1 and/or M3 antagonist in combination with one or more pharmaceutically active agents as set forth herein. For instance, the formulation may contain a selective muscarinic acetylcholine receptor subtype M1 and/or M3 antagonist in combination with desloratadine and/or pseudoephedrine for the treatment of the referenced disorder (e.g., a respiratory disorder associated with the production of mucus glycoprotein, a skin disorder, or allergic conjunctivitis).

The formulations of the present invention may be administered in specific, measured amounts in the form an aerosol (solution and/or suspension). The aerosol may be delivered using devices found useful for providing measured substantially non-systematically bioavailable amounts of aerosolized pharmaceutical compositions thereof for delivery to the oral airway passages and lungs by oral inhalation or intranasally by inhalation. Such devices include pressurized metered-dose inhalers (“MDI”) which deliver aerosolized particles suspended in chlorofluorocarbon propellants (e.g., CFC-11, CFC-12), or the non-chlorofluorocarbons or alternate propellants (e.g., the fluorocarbons, HFC-134A or HFC-227) with or without surfactants and suitable bridging agents.

The formulations of the present invention may be also administered in specific, measured amounts in the form of an aqueous suspension by use of a pump spray bottle. Such pump spray bottles include the pump spray bottle used to deliver NASONEX® Nasal Spray as well as the pump spray bottle disclosed in the Schering Corporation Industrial Design Deposit DM/026304, registered by the Hague Union on Jun. 1, 1993 (each are available from Schering Corporation).

The formulations of the present invention may also be administered via a Dry Powder Inhaler. Such inhalers include Schering's Twisthaler, Diskhaler (Allen & Hanburys), Accuhaler (Allen & Hanburys), Diskus (Glaxo), Spiros (Dura), Easyhaler (Orion), Cyclohaler (Pharmachemie), Cyclovent (Pharmachemie), Rotahaler (Glaxo), Spinhaler (Fisons), FlowCaps(Hovione), Turbospin (PH&T), Turbohaler (Astra), EZ Breath (Norton Healthcare/IVAX), MIAT-HALER (Miat), Pulvinal (Chiesi), Ultrahaler (Fisons/Rhone Poulenc Rorer), MAG-Haler (GGU), Prohaler (Valois), Taifun (Leiras), JAGO DPI (JAGO), and M L Laboratories' DPI (M L Laboratories).

For oral dosage form preparations, a pharmaceutically acceptable carrier (which includes diluents, excipients, or carrier materials) is also present in the formulation. The carrier is suitably selected with respect to the intended form of administration, i.e., oral tablets, capsules (either solid-filled, semi-solid filled, or liquid filled), powders for constitution, oral gels, orally consumable films, elixirs, syrups, suspensions, and the like, and consistent with conventional pharmaceutical practices. For example, for oral administration in the form of tablets or capsules, the medicinal agent may be combined with any oral non-toxic pharmaceutically acceptable inert carrier, such as lactose, starch, sucrose, cellulose, magnesium stearate, dicalcium phosphate, calcium sulfate, mannitol, ethyl alcohol (liquid forms), and the like. Moreover, when desired or needed, suitable binders, lubricants, disintegrants, disinfectants and coloring agents may also be incorporated in the mixture. Suitable binders include starch, gelatin, natural sugars, corn sweeteners, natural and synthetic gums such as acacia, sodium alginate, carboxymethylcellulose, polyethylene glycol, and waxes. Suitable lubricants include boric acid, sodium benzoate, sodium acetate, sodium chloride, and the like. Suitable disintegrants include starch, methylcellulose, guar gum, and the like. Suitable disinfectants include benzalkonium chloride and the like. Sweetening and flavoring agents and preservatives may also be included where appropriate.

Additionally, the formulations of the present invention may be formulated in sustained release form to provide the rate controlled release of any one or more of the components or medicinal agents to optimize the therapeutic effects. Suitable dosage forms for sustained release include layered tablets (e.g., containing layers of varying disintegration rates or controlled release polymeric matrices impregnated with the medicinal agents) that are shaped in tablet form or capsules containing such impregnated or encapsulated porous polymeric matrices.

Conventional methods for preparing tablets are known. Such methods include dry methods such as direct compression and compression of granulation produced by compaction, wet methods, or other special procedures.

For ophthalmic compositions, the compositions of the present invention may take various forms. For example, they may be an aqueous gel or liquid, or an ointment. In a preferred embodiment, the composition is a water-in-oil emulsion with the active ingredients in the aqueous droplets suspended in a lotion or flowable ointment base comprising, e.g., petrolatum, mineral oil, and the like. Additional emollient ingredients such as isopropyl myristate may also be added. Such a lotion or ointment covers the conjunctiva and cornea with a thin film that both carries active ingredients and provides for prolonged drainage through the naso-lacrimal ducts. The film also provides a barrier to evaporative loss of water from the corneal stroma.

The selective muscarinic acetylcholine receptor subtype M1 and/or M3 antagonist and one or more pharmaceutically active agents administered in the method of treating the referenced disorder (e.g., a respiratory disorder associated with the production of mucus glycoprotein, a skin disorder, or allergic conjunctivitis) may be administered concurrently or sequentially (i.e., by the sequential administration of the ingredients in a suitable order).

The formulations disclosed herein may be either sedating or non-sedating. In one embodiment, the formulations disclosed herein are sedating. In an alternative embodiment, the formulations disclosed herein are non-sedating or mildly sedating.

For disorders of the lower airways, the severity in a patient can be quantified by objective pulmonary function tests, including a measurement of the patient's forced expiratory volume in 1 second (FEV₁). When this result is about 65 to 79 percent of the predicted value (determined using a formula that takes into account the patient's age and size), the airway obstruction is considered to be mild. For an FEV, value about 50 to 64 percent of predicted, the airway obstruction is classified as moderate; if the value is less than 50 percent of predicted, the airway obstruction is considered to be severe; and if the value is less than 30 percent the airway obstruction is considered to be very severe. This test utilizes relatively simple and inexpensive equipment, and therefore is widely used for diagnosis, and to monitor the progression lung and airway disorders during treatment.

For disorders of the upper airways, there are also objective parameters for measuring an improvement in symptoms. Efficacy endpoints studies may include Total Symptom Score, Total Nasal Symptom Score, Total Non-nasal Symptom Score, Individual Symptom Scores, and Health Quality of Life (HQOL) analysis in efficacy trials. The compositions of the present invention may be tested for reducing the total symptom scores (the sum of individual scores for rhinorrhea, post-nasal drip, sneezing, congestion/stuffiness, nasal itching, itchy/burning eyes, tearing, ocular redness, and itchy ears/palate). An important efficacy endpoint that may be analyzed in the studies is the AM NOW total symptom score. This parameter measures the total symptom relief by the patient after 24 hours before taking the next day dose.

The compositions of the present invention may be particularly useful for the treatment and prevention of the nasal (stuffiness/congestion, rhinorrhea, post-nasal drip, nasal itching, sneezing) and non-nasal (itchy/burning eyes, tearing/watery eyes, redness of the eyes, itching of the ears/palate) symptoms of seasonal and perennial allergic rhinitis, including nasal congestion, in patients in need of such treating and/or preventing.

### EXAMPLE

This is a Phase II, randomized, double-blind, placebo controlled, double-dummy, multicenter, dose-ranging study of desloratadine (DL) in subjects at least 18 years of age with Seasonal Allergic Rhinitis (SAR) and Post-Nasal Drip. Subjects were randomized to one of 5 treatment arms: DL 2.5 mg BID, Oxybutynin (Oxy) 5 mg BID, DL 2.5 mg+Oxy 2.5 mg given concurrently BID, DL 2.5 mg +Oxy 5 mg given concurrently BID, or placebo in a 1:1:1:1:1 ratio. The total target enrollment was 500 subjects with 100 per arm. The study consisted of a 7-day Treatment Phase.

The primary efficacy variable was the change from baseline in the average AM/PM PRIOR post nasal drip score over the Treatment Phase of 7 days. The analysis was based on a main effect analysis of variance (ANOVA), which extracted sources of variation due to treatment and center. The primary objective is to estimate the effect of DL 2.5 mg+Oxy 5 mg in reducing post nasal drip compared to DL alone and placebo. Pairwise treatment comparisons were examined using the 95% confidence intervals of the treatment differences at an unadjusted 2-sided alpha-level of 0.05.

The order of examination was as follows: DL 2.5 mg+Oxy 5 mg BID vs. DL and DL 2.5 mg+Oxy 5 mg BID vs. placebo, followed by DL 2.5 mg+Oxy 2.5 mg BID vs. DL and DL 2.5 mg+Oxy 2.5 mg BID vs. placebo.

After the examination of post nasal drip, the key secondary efficacy variable, anterior rhinorrhea, was examined in the same manner.

A preliminary assessment of the consistency of results across centers for the primary efficacy variable was made using an ANOVA model that extracted sources of variation due to treatment, center, and treatment-by-center interaction. To be included in this analysis, centers must have had at least two subjects in each treatment group with post nasal drip evaluations.

There were 540 subjects randomized into the study. Of those, 106 subjects were in the DL 2.5 mg BID group; 108, 108, 111 and 107 were in the Oxy 5 mg BID, DL 2.5 mg+Oxy 2.5 mg BID, DL 2.5 mg+Oxy 5 mg BID and placebo groups, respectively. The table below provides a summary of the numbers of subjects in the analysis subsets.

| | | | | DL | | |
|---|---|---|---|---|---|---|
| | DL 2.5 | Oxy 5 | DL2.5/ | 2.5/Oxy | | |
| Subjects | BID | mg bID | Oxy2.5bid | 5 bid | Placebo | Total |
| Randomized | 106 | 108 | 108 | 111 | 107 | 540 |
| Efficacy | 999 | 97 | 100 | 105 | 101 | 502 |

Of the 540 randomized subjects, 384 (71.11%) were female, 156 (28.89%) were male; 421 (77.96%) were Caucasian, 119 (22.04%) were non-Caucasian. Subjects' age ranged from 18 to 76 years.

There were 38 subjects excluded from the efficacy evaluable subset. A majority of these subjects were excluded due to insufficient efficacy data. Determination of evaluability was perfomed prior to unblinding the database. Though the treatment duration specified in the protocol was 7 days, about 70% of subjects also completed their diary on the AM of Day 8 since the final office visit was scheduled on that day. Therefore, the AM Day 8 data were included in the calculation of the one-week average.

Overall, only 14 (2.6%) subjects discontinued from the treatment phase. Most of them occurred in the Oxy 5 mg BID group (5.56%), while the other four treatment groups (DL 2.5 mg, DL 2.5 mg+Oxy 2.5 mg, DL 2.5 mg+Oxy 5 mg, and placebo) had a discontinuation rate of 1.89%, 0.93%, 1.80% and 2.80% respectively. Discontinuation due to adverse events (6, 1.11%) was the most common reason for discontinuation across the treatment groups.

The primary efficacy variable was the change from baseline in the average AM/PM PRIOR post nasal drip score over the Treatment Phase of 7 days.

The Baseline post nasal drip scores were comparable across the treatment groups, ranging from 2.60 in the DL 2.5 mg+Oxy 2.5 mg BID treatment to 2.66 in placebo. The least square mean changes were −0.68(−24.8%), −0.49 (−17.8%), −0.60(−22.2%)−0.63 (−23.8%), and −0.57 (−20.7%), in the DL 2.5 mg BID, Oxy 5 mg BID, DL 2.5 mg+Oxy 2.5 mg BID, DL 2.5 mg+Oxy 5 mg BID and placebo respectively.

The treatment difference between DL 2.5 mg+Oxy 5 mg BID and DL alone was 0.05 point in favor of DL alone. In addition, the difference between this combination and placebo is 0.06 points and represents only ¼ of the target difference (0.24 points) specified in the protocol. The 95% confidence interval for the difference between DL 2.5 mg+Oxy 5 mg BID and DL alone was between −0.21 and 0.10. A confirmatory analysis based on the evaluable subjects was consistent with the primary results based on all randomized subjects.

There was evidence of a slight treatment-by-center interaction (p=0.13) in a full-model analysis of variance, indicating that the above results might vary across centers. After further exploration, evidence of this interaction can be attributed to the Oxy 5 mg BID and placebo treatment groups.

The Oxy 5 mg BID treatment demonstrated a −0.49 least squares mean decrease from baseline which was less than the placebo response.

The key secondary efficacy variable, anterior rhinorrhea, was also examined. The combination of DL 2.5 mg+Oxy 5 mg demonstrated a treatment difference of 0.19 points vs. placebo and 0.16 points vs. DL alone. The lower dose combination of DL 2.5 mg+Oxy 2.5 mg BID demonstrated a 0.14 point advantage over placebo, and a 0.11 point advantage over DL alone. However, the differences between placebo and the individual components DL and Oxy alone were only 0.03 and 0.04, respectively.

The results for the total symptom score excluding post nasal drip, demonstrated a treatment difference of 0.71 point between DL alone and placebo. This is similar to the results from previous studies of DL in subjects with SAR, confirming the activity of DL in this study.

The percentage of subjects reporting any treatment emergent adverse events was 22.78% (123/540 subjects) overall. Of these, 17 subjects (16.0%) were in the DL 2.5 mg group, 33 (30.6%), 22 (20.4%), 36 (32.4%) and 15 (14.0%) subjects were in the Oxy 5 mg BID , DL 2.5 mg+Oxy 2.5 mg BID, DL 2.5 mg+Oxy 5 mg BID, and placebo groups respectively. Dry mouth was the most common adverse event, reported by 49 (9.07%) subjects across the 5 treatment groups. There were 15 subjects reporting dry mouth in the Oxy 5 mg BID group (13.9%), and 19 subjects reporting the events in the DL 2.5 mg+Oxy 5 mg group (17.1%). The lower dose combination of DL 2.5 mg+Oxy 2.5 mg BID group had 8 subjects (7.4%) reporting dry mouth. These rates are higher than that of DL alone with 4 subjects (3.8%) and placebo with 3 subjects (2.8%).

Except for dry mouth, similar adverse event profiles were noted across the five treatments.

The differences between DL 2.5 mg+Oxy 5 mg BID and placebo or DL alone in the treatment of post nasal drip were 0.06 and 0.05, respectively, and did not approach the target difference of 0.24 points. Even though the lower limits of the 95% confidence intervals (−0.21 and −0.20) did allow for the possibility of approaching the target treatment difference, the probability of observing such differences (0.06 and 0.05) are low if the true treatment difference was 0.17.

However, for anterior rhinorrhea, the key secondary endpoint, the differences between DL 2.5 mg+Oxy 5 mg BID and placebo or DL alone were near or above the least significant difference of 0.17 points. The incidence rates of dry mouth in the three Oxy-containing treatment groups were greater than that of DL alone or placebo, consistent with the adverse event profile of Oxybutynin.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims. Various publications are cited herein, the disclosures of which are incorporated by reference in their entireties.

What is claimed:

1. A formulation comprising a therapeutically effective amount of oxybutynin or a pharmaceutically acceptable salt thereof in combination with a therapeutically effective amount of desloratadine or a pharmaceutically acceptable salt thereof.

2. The formulation of claim 1 wherein the pharmaceutically acceptable salt of oxybutynin is prepared from a pharmaceutically acceptable acid addition salt selected from the group consisting of acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, and p-toluene sulfonic acid.

3. The formulation of claim 1 wherein the pharmaceutically acceptable salt of oxybutynin is oxybutynin chloride.

4. The formulation of claim 1 wherein the therapeutically effective amount of oxybutynin or a pharmaceutically acceptable salt thereof is a unit dosage form in a range from about 0.1 mg to about 1 g administered q.d.

5. The formulation of claim 1 wherein the therapeutically effective amount of oxybutynin or a pharmaceutically acceptable salt thereof is a unit dosage form in a range from about 1 mg to about 1 g administered q.d.

6. The formulation of claim 1 wherein the therapeutically effective amount of oxybutynin or a pharmaceutically acceptable salt thereof is a unit dosage form in a range from about 25 mg to about 700 mg administered q.d.

7. The formulation of claim 1 wherein the therapeutically effective amount of oxybutynin or a pharmaceutically acceptable salt thereof is a unit dosage form in a range from about 0.1 mg to about 100 mg administered q.d.

8. The formulation of claim 1 wherein the therapeutically effective amount of oxybutynin or a pharmaceutically acceptable salt thereof is a unit dosage form in a range from about 1.25 mg to about 30 mg administered q.d.

9. The formulation of claim 1 wherein the therapeutically effective amount of oxybutynin or a pharmaceutically acceptable salt thereof is a unit dosage form which is about 1.25 mg, about 2.5 mg, about 5 mg, about 10 mg, about 15 mg, about 20 mg, about 25 mg, or about 30 mg administered q.d.

10. The formulation of claim 1 wherein the therapeutically effective amount of oxybutynin or a pharmaceutically acceptable salt thereof is a unit dosage form which is about 1.25 mg, about 2.5 mg, about 3 mg, about 3.5 mg, about 4 mg, about 4.5 mg, or about 5 mg and which is administered b.i.d., t.i.d., or q.i.d.

11. The formulation of claim 1 wherein the therapeutically effective amount of oxybutynin or a pharmaceutically acceptable salt thereof is a unit dosage form which is about 2.5 mg and which is administered b.i.d.

12. The formulation of claim 1 wherein the therapeutically effective amount of oxybutynin or a pharmaceutically acceptable salt thereof is a unit dosage form which is about 5 mg and which is administered b.i.d.

13. The formulation of claim 1 wherein the therapeutically effective amount of desloratadine or a pharmaceutically acceptable salt thereof is a unit dosage form in a range from about 1.25 mg to about 45 mg administered q.d.

14. The formulation of claim 1 wherein the therapeutically effective amount of desloratadine or a pharmaceutically acceptable salt thereof is a unit dosage form in a range from about 1.25 mg to about 20 mg administered q.d.

15. The formulation of claim 1 wherein the therapeutically effective amount of desloratadine or a pharmaceutically acceptable salt thereof is a unit dosage form in a range from about 5 mg to about 10 mg administered q.d.

16. The formulation of claim 1 wherein the therapeutically effective amount of desloratadine or a pharmaceutically acceptable salt thereof is a unit dosage form which is about 1.25 mg, about 2.5 mg, about 5.0 mg, about 7.5 mg, about 10.0 mg, about 12.5 mg, about 15 mg, 17.5 mg, or about 20.0 mg administered q.d.

17. The formulation of claim 1 wherein the therapeutically effective amount of desloratadine or a pharmaceutically acceptable salt thereof is a unit dosage form which is about 1.25 mg administered q.d.

18. The formulation of claim 1 wherein the therapeutically effective amount of desloratadine or a pharmaceutically acceptable salt thereof is a unit dosage form which is about 2.5 mg administered q.d.

19. The formulation of claim 1 wherein the therapeutically effective amount of desloratadine or a pharmaceutically acceptable salt thereof is a unit dosage form which is about 5.0 mg administered q.d.

20. The formulation of claim 1 wherein the therapeutically effective amount of desloratadine or a pharmaceutically acceptable salt thereof is a unit dosage form which is about 10.0 mg administered q.d.

21. The formulation of claim 1 wherein the therapeutically effective amount of desloratadine or a pharmaceutically acceptable salt thereof is a unit dosage form which is about 20.0 mg administered q.d.

22. The formulation of claim 1 further comprising one or more additional pharmaceutically active agents.

23. The formulation of claim 22 wherein one or more additional pharmaceutically active agents is another antihistamine.

24. The formulation of claim 22 wherein one or more additional pharmaceutically active agents is a decongestant.

25. The formulation of claim 22 wherein one or more additional pharmaceutically active agents is a corticosteroid.

26. The formulation of claim 22 wherein one or more additional pharmaceutically active agents is an expectorant.

27. The formulation of claim 22 wherein one or more additional pharmaceutically active agents is a composition to relieve oropharyngeal discomfort.

28. The formulation of claim 22 wherein one or more additional pharmaceutically active agents is a P2Y₂ receptor antagonist.

29. The formulation of claim 22 wherein one or more additional pharmaceutically active agents is a non-steroidal anti-inflammatory agent.

30. The formulation of claim 22 wherein one or more additional pharmaceutically active agents is a leukotriene antagonist.

31. The formulation of claim 22 wherein one or more additional pharmaceutically active agents is a syk kinase inhibitor.

32. The formulation of claim 22 wherein one or more additional pharmaceutically active agents is a 5-lipoxygenase inhibitor.

33. A method for treating a respiratory disorder associated with the production of mucus glycoprotein in a patient suffering therefrom comprising administering a therapeutically effective amount of oxybutynin or a pharmaceutically acceptable salt thereof in combination with a therapeutically effective amount of desloratadine or a pharmaceutically acceptable salt thereof.

34. The method of claim 33 wherein the pharmaceutically acceptable salt of oxybutynin is prepared from a pharmaceutically acceptable acid addition salt selected from the group consisting of acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, and p-toluene sulfonic acid.

35. The method of claim 33 wherein the pharmaceutically acceptable salt of oxybutynin is oxybutynin chloride.

36. The method of claim 33 wherein the therapeutically effective amount of oxybutynin or a pharmaceutically acceptable salt thereof is a unit dosage form in a range from about 0.1 mg to about 1 g administered q.d.

37. The method of claim 33 wherein the therapeutically effective amount of oxybutynin or a pharmaceutically acceptable salt thereof is a unit dosage form in a range from about 1 mg to about 1 g administered q.d.

38. The method of claim 33 wherein the therapeutically effective amount of oxybutynin or a pharmaceutically acceptable salt thereof is a unit dosage form in a range from about 25 mg to about 700 mg administered q.d.

39. The method of claim 33 wherein the therapeutically effective amount of oxybutynin or a pharmaceutically acceptable salt thereof is a unit dosage form in a range from about 0.1 mg to about 100 mg administered q.d.

40. The method of claim 33 wherein the therapeutically effective amount of oxybutynin or a pharmaceutically acceptable salt thereof is a unit dosage form in a range from about 1.25 mg to about 30 mg administered q.d.

41. The method of claim 33 wherein the therapeutically effective amount of oxybutynin or a pharmaceutically acceptable salt thereof is a unit dosage form which is about 1.25 mg, about 2.5 mg, about 3 mg, about 3.5 mg, about 4 mg, about 4.5 mg, or about 5 mg administered q.d.

42. The method of claim 33 wherein the therapeutically effective amount of oxybutynin or a pharmaceutically acceptable salt thereof is a unit dosage form which is about 1.25 mg, about 2.5 mg, about 3 mg, about 3.5 mg, about 4 mg, about 4.5 mg, or about 5 mg and which is administered b.i.d., t.i.d., or q.i.d.

43. The method of claim 33 wherein the therapeutically effective amount of oxybutynin or a pharmaceutically acceptable salt thereof is a unit dosage form which is about 2.5 mg and which is administered b.i.d.

44. The method of claim 33 wherein the therapeutically effective amount of oxybutynin or a pharmaceutically acceptable salt thereof is a unit dosage form which is about 5 mg and which is administered b.i.e.

45. The method of claim 33 wherein the therapeutically effective amount of desloratadine or a pharmaceutically acceptable salt thereof is a unit dosage form in a range from about 1.25 mg to about 45 mg administered q.d.

46. The method of claim 33 wherein the therapeutically effective amount of desloratadine or a pharmaceutically acceptable salt thereof is a unit dosage form in a range from about 1.25 mg to about 20 mg administered q.d.

47. The method of claim 33 wherein the therapeutically effective amount of desloratadine or a pharmaceutically acceptable salt thereof is a unit dosage form in a range from about 5 mg to about 10 mg administered q.d.

48. The method of claim 33 wherein the therapeutically effective amount of desloratadine or a pharmaceutically acceptable salt thereof is a unit dosage form which is about 1.25 mg, about 2.5 mg, about 5.0 mg, about 7.5 mg, about 10.0 mg, about 12.5 mg, about 15 mg, 17.5 mg, or about 20.0 mg administered q.d.

49. The method of claim 33 wherein the therapeutically effective amount of desloratadine or a pharmaceutically acceptable salt thereof is a unit dosage form which is about 1.25 mg administered q.d.

50. The method of claim 33 wherein the therapeutically effective amount of desloratadine or a pharmaceutically acceptable salt thereof is a unit dosage form which is about 2.5 mg administered q.d.

51. The method of claim 33 wherein the therapeutically effective amount of desloratadine or a pharmaceutically acceptable salt thereof is a unit dosage form which is about 5.0 mg administered q.d.

52. The method of claim 33 wherein the therapeutically effective amount of desloratadine or a pharmaceutically acceptable salt thereof is a unit dosage form which is about 10.0 mg administered q.d.

53. The method of claim 33 wherein the therapeutically effective amount of desloratadine or a pharmaceutically acceptable salt thereof is a unit dosage form which is about 20.0 mg administered q.d.

54. The method of claim 33 further comprising administering one or more additional pharmaceutically active agents.

55. The method of claim 54 wherein one or more additional pharmaceutically active agents is another antihistamine.

56. The method of claim 54 wherein one or more additional pharmaceutically active agents is a decongestant.

57. The method of claim 54 wherein one or more additional pharmaceutically active agents is a corticosteroid.

58. The method of claim 54 wherein one or more additional pharmaceutically active agents is an expectorant.

59. The method of claim 54 wherein one or more additional pharmaceutically active agents is a composition to relieve oropharyngeal discomfort.

60. The method of claim 54 wherein one or more additional pharmaceutically active agents is a P2Y₂ receptor antagonist.

61. The method of claim 54 wherein one or more additional pharmaceutically active agents is a non-steroidal anti-inflammatory agent.

62. The method of claim 54 wherein one or more additional pharmaceutically active agents is a leukotriene antagonist.

63. The method of claim 54 wherein one or more additional pharmaceutically active agents is a syk kinase inhibitor.

64. The method of claim 54 wherein one or more additional pharmaceutically active agents is a 5-lipoxygenase inhibitor.

65. A method for treating a skin disorder in a patient suffering therefrom comprising administering a therapeutically effective amount of oxybutynin or a pharmaceutically acceptable salt thereof in combination with a therapeutically effective amount of desloratadine or a pharmaceutically acceptable salt thereof.

66. A method for treating allergic conjunctivitis in a patient suffering therefrom comprising administering a therapeutically effective amount of oxybutynin or a pharmaceutically acceptable salt thereof in combination with a therapeutically effective amount of desloratadine or a pharmaceutically acceptable salt thereof.

67. A formulation for the treatment of anterior rhinorrhea comprising a therapeutically effective amount of oxybutynin or a pharmaceutically acceptable salt thereof in combination with a therapeutically effective amount of desloratadine or a pharmaceutically acceptable salt thereof.

68. The formulation of claim 67 further comprising a second pharmaceutically active agent.

69. The formulation of claim 68 wherein one or more additional pharmaceutically active agents is another antihistamine.

70. The formulation of claim 68 wherein one or more additional pharmaceutically active agents is a decongestant.

71. The formulation of claim 68 wherein one or more additional pharmaceutically active agents is a corticosteroid.

72. The formulation of claim 68 wherein one or more additional pharmaceutically active agents is an expectorant.

73. The formulation of claim 68 wherein one or more additional pharmaceutically active agents is a composition to relieve oropharyngeal discomfort.

74. The formulation of claim 68 wherein one or more additional pharmaceutically active agents is a P2Y₂ receptor antagonist.

75. The formulation of claim 68 wherein one or more additional pharmaceutically active agents is a non-steroidal anti-inflammatory agent.

76. The formulation of claim 68 wherein one or more additional pharmaceutically active agents is a leukotriene antagonist.

77. The formulation of claim 68 wherein one or more additional pharmaceutically active agents is a syk kinase inhibitor.

78. The formulation of claim 68 wherein one or more additional pharmaceutically active agents is a 5-lipoxygenase inhibitor.
